(19) United States
(12) Patent Application Publication
(10) Pub. No.: US 2024/0110142 A1
(43) Pub. Date: Apr. 4, 2024

(54) ASEPTIC CELL CULTURE SYSTEM AND METHODS OF USE

(71) Applicant: Life Technologies Corporation, Carlsbad, CA (US)

(72) Inventors: Mark Thomas Smith, Nibley, UT (US); David Klinkenberg, Copenhagen (DK)

(21) Appl. No.: 18/474,004

(22) Filed: Sep. 25, 2023

Related U.S. Application Data
(60) Provisional application No. 63/377,937, filed on Sep. 30, 2022.

(51) Int. Cl. C12M 1/00; C12M 1/26

(52) U.S. Cl. CPC C12M 23/58; C12M 33/14

(57) ABSTRACT

An aseptic cell culture system including a cell culture device is disclosed. The cell culture device includes an interior chamber having a first opening extending through a wall defining a part of the interior chamber, and a first aseptic port. The first aseptic port includes a first coupling end and a second coupling end, the first coupling end extending through the first opening and integrated with the wall. Further, the first aseptic port includes a fluid pathway in fluid communication with the interior chamber and extends between the first coupling end and the second coupling end. The second coupling end includes a connecting component configured to allow one or more of a fluid line and a filter to be connected to the first aseptic port.

### CROSS-REFERENCE TO RELATED APPLICATIONS

This application claims the benefit of U.S. Provisional Application No. 63/377,937, filed on Sep. 30, 2022, which is incorporated herein by specific reference.

### BACKGROUND

Cell culture systems, including stacked cell culture trays, are useful for large-scale cell cultivation and have become popular as an alternative to conventional roller bottles, bioreactors, and the like. Exemplary stacked tray systems include the Thermo Scientific™ Nunc™ Cell Factory™ systems, the Corning® CELLSTACK® (Corning Inc., Lowell, Mass.) and the Millipore® MILLICELL® HY flasks (Millipore Corp., Billerica, Mass.). Such systems include one or more cell culture trays.

Maintaining the sterile integrity of cell culture systems can be difficult, such as when transporting cell culture systems, adapting the cell culture system to various components, such as tubing (e.g., fluid lines), and transferring fluid in and out of the cell culture system. Efforts to seal cell culture systems to prevent contamination can result in deformation and damage to the cell culture system, such as due to the cell culture system being unable to effectively vent to achieve pressure equilibration within the cell culture system. Such inability to effectively vent can cause pressure levels in the cell culture system to reach a level that causes damage to the structural integrity of the cell culture system (e.g., brake fluid seals, etc.), which can result in the cell culture system becoming unusable. However, if the cell culture system is not properly protected to prevent contamination, added time and cost is required to certify sterile conditions of the cell culture system prior to use. As such, cost and time can be lost for both users and manufacturers due to sterility and venting requirements necessary to use and operate cell culture systems.

Other issues associated with maintaining the sterile integrity and aseptic environment within cell culture systems include adapting various components to cell culture systems without disrupting a sterile or aseptic environment within the cell culture system. For example, it may be desired to introduce a fluid into the cell culture system, however, several steps are required to achieve sterile and aseptic conditions when coupling fluid lines and transferring fluid to and from cell culture systems, which can increase time and costs for the user. As such, improved cell culture systems are desired.

### SUMMARY

Aspects of the current subject matter include various embodiments and related methods of an aseptic cell culture system. In one aspect, an aseptic cell culture system is described that includes a cell culture device including an interior chamber and a first opening extending through a wall defining a part of the interior chamber. The aseptic cell culture system can also include a first aseptic port having a first coupling end and a second coupling end. The first coupling end can extend through the first opening and be integrated with the wall. The first aseptic port can include a fluid pathway in fluid communication with the interior chamber and extend between the first coupling end and the second coupling end. The second coupling end can include a connecting component configured to allow one or more of a fluid line and a filter to be connected to the first aseptic port.

In some variations one or more of the following features can optionally be included in any feasible combination. The first aseptic port can include a membrane positioned across a cross-section of the fluid pathway, and the membrane can be configured to prevent contaminants from passing through the membrane. The membrane can be positioned along a planar surface of the second coupling end and cover a distal end of the fluid pathway. The connecting component can be positioned along the planar surface, and the connecting component and the planar surface can form at least a part of a first genderless connecting interface of the first aseptic port.

In some embodiments, the aseptic cell culture system can further include a connecting port having a second genderless connecting interface that couples with the first genderless connecting interface of the first aseptic port. The connecting port can include a connector fluid pathway that is in fluid communication with the fluid pathway of the first aseptic port when the connecting port is coupled to the first aseptic port. The connecting port can include a tubing connector configured to couple to a tubing for allowing fluid to flow into the interior chamber or out of the interior chamber when the connecting port is coupled to the first aseptic port. The fluid can be one or more of a gas, air, liquid, and cell culture medium. The connecting port can include a filter positioned along the connector fluid pathway for filtering gas and/or air flowing into and/or out of the first aseptic port connected to the connecting port. The second coupling end can form a locked engagement with the wall to prevent uncoupling of the first aseptic port from the cell culture device. The locked engagement can be formed by one or more of an adhesive and a mechanical coupling between the first aseptic port and the cell culture device. In some embodiments, the locked engagement is releasable. In some embodiments, the locked engagement is a snap-fit engagement.

In some embodiments, the first aseptic port, the membrane, and the cell culture device can be formed of a material that withstands gamma-irradiation of at least 50 kilogray (kGy). The cell culture device can include a second aseptic port extending through a second opening of the cell culture device thereby providing a second fluid pathway for liquid and/or gas to flow either in or out of the interior chamber. The cell culture device can be a multi-layer cell growth system comprising a plurality of cell culture trays.

In another aspect, a method of manufacturing an aseptic cell culture system is described that includes coupling a first aseptic port to a first opening of a cell culture device to form the aseptic cell culture system. The coupling can include forming a locked engagement and a fluidic seal between the first aseptic port and the cell culture device. Additionally, the method can include sterilizing the aseptic cell culture system.

In some variations one or more of the following can optionally be included in any feasible combination. The sterilizing can include one or more of irradiation, autoclave, ethylene oxide, chemical disinfectants, or dry heat sterilization. The sterilizing can include gamma-irradiation of at least 50 kGy. The method can further include coupling a second aseptic port to a second opening of the cell culture device. The method can further include coupling a membrane to the first aseptic port such that the membrane is positioned over a cross-section of the fluid pathway to prevent contaminants from entering an interior chamber of the cell culture device.

In another aspect, an aseptic cell culture system kit is described. The aseptic cell culture system kit can include an aseptic cell culture system including a cell culture device including an interior chamber and a first opening extending through a wall defining a part of the interior chamber. The cell culture device can further include a first aseptic port including a first coupling end and a second coupling end. The first coupling end can extend through the first opening and be integrated with the wall. The first aseptic port can include a fluid pathway in fluid communication with the interior chamber and extend between the first coupling end and the second coupling end. The second coupling end can include a connecting component configured to allow one or more of a fluid line and a filter to be connected to the first aseptic port. The aseptic cell culture system kit can further include a plurality of connecting ports that are each configured to connect to the first aseptic port.

In some variations one or more of the following features can optionally be included in any feasible combination. The first aseptic port can include a membrane positioned across a cross-section of the fluid pathway, and the membrane can be configured to prevent contaminants from passing through the membrane. The first aseptic port can include a first genderless connecting interface. Each connecting port of the plurality of connecting ports can include a second genderless connecting interface configured to couple to the first genderless connecting interface of the first aseptic port. A first connecting port of the plurality of connecting ports can include a tubing connector configured to couple to a tubing. A second connecting port of the plurality of connecting ports can include a filter. The second coupling end of the first aseptic port can form a locked engagement with the wall to prevent uncoupling of the first aseptic port from the cell culture device. The locked engagement can be formed by one or more of an adhesive and a mechanical coupling between the first aseptic port and the cell culture device. The cell culture device can include a second aseptic port integrated with the cell culture device, and each of the plurality of connecting ports can be configured to couple to the first aseptic port and the second aseptic port. The cell culture device can be a multi-layer cell growth system including a plurality of cell culture trays.

In another aspect, a method of an aseptic cell culture system includes introducing a cell culture medium to an interior chamber of a cell culture device of the cell culture system. The cell culture medium can be introduced through an aseptic port integrated with the cell culture device. The aseptic port can include a first coupling end that is integrated with the cell culture device, and a fluid pathway that is in fluid communication with the interior chamber and extends between the first coupling end and a second coupling end. The second coupling end can include a connecting component configured to allow one or more of a fluid line and a filter to be connected to the aseptic port. The method can also include removing, after processing the cell culture medium in the interior chamber, a volume of cell material from the aseptic cell culture system. The method can further include disposing, after removal of the volume of cell material, the aseptic cell culture system.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, illustrate embodiments of the invention and, together with the general description given above and the detailed description of the embodiments given below, serve to explain the principles of the present invention. In the figures, corresponding or like numbers or characters indicate corresponding or like structures.

FIG. 1 is a perspective view showing a cell culture device in accordance with embodiments disclosed herein.

FIG. 2 is an enlarged partial perspective view of a venting port of the cell culture device of FIG. 1.

FIG. 3 is a cross-sectional view of a portion of the cell culture device of FIG. 1, taken along the line 3-3 in FIG. 1.

FIG. 4 is a top perspective view of an aseptic cell culture system in accordance with embodiments disclosed herein.

FIG. 5A is a side view of an embodiment of an aseptic port of the aseptic cell culture system of FIG. 4.

FIG. 5B is a cross-sectional view of the aseptic port of FIG. 5A taken along the line 5B-5B in FIG. 5A.

FIG. 5C is a cross-sectional view of a portion of the aseptic cell culture system of FIG. 4 showing a first coupling end of the aseptic port integrated with the cell culture device.

FIG. 5D is a side view of another embodiment of the aseptic port including a tubing connector or barb.

FIG. 5E is a cross-sectional view of the aseptic port of FIG. 5D taken along the line 5E-5E in FIG. 5D.

FIG. 5F is a section view of the aseptic port of FIG. 5D showing features of the connecting component.

FIG. 6A is a top perspective view of the aseptic port prior to integrating with the cell culture device to form the aseptic cell culture system of FIG. 4.

FIG. 6B is a top perspective view of an embodiment of the aseptic cell culture system including a first aseptic port integrated with a first venting port, a second aseptic port integrated with a second venting port, and a connecting port configured to connect to a tubing and the first or second aseptic port.

FIG. 6C is a top perspective view of an embodiment of the aseptic cell culture system including the first aseptic port integrated with the first venting port and coupled to the connecting port, and the second aseptic port integrated with the second venting port.

FIG. 6D is a top perspective view of an embodiment of the aseptic cell culture system including the first aseptic port integrated with the first venting port and coupled to the first connecting port, and the second aseptic port integrated with the second venting port and coupled to a second connecting port.

FIG. 6E is a top perspective view of an embodiment of the aseptic cell culture system including the first aseptic port integrated with the first venting port, the second aseptic port integrated with the second venting port, the first connecting port including a tubing for connecting to the first aseptic port, and a second connecting port including a filter.

FIG. 6F is a side perspective view of an embodiment of the aseptic cell culture system including the first aseptic port coupled to the first connecting port including the tubing, and the second aseptic port coupled to the second connecting port.

FIG. 7A is a top perspective view of an embodiment of a connecting port including a filter housing.

FIG. 7B is an exploded top perspective view of the connecting port of FIG. 7A.

FIG. 7C is a side view of the connecting port of FIG. 7A.

### DETAILED DESCRIPTION

Disclosed herein are systems and methods of an aseptic cell culture system that can efficiently and effectively maintain a sterile and/or aseptic environment in the aseptic cell culture system. The aseptic cell culture system is also configured to efficiently and effectively allow the aseptic cell culture system to breathe and achieve pressure equilibrium, such as to prevent against structural damage to the structure and/or contents of the aseptic cell culture system. The aseptic cell culture system can also allow for efficient and effective coupling to various components, such as tubing (e.g., fluid lines) and filters while maintaining an aseptic environment in the aseptic cell culture system. As such, components of the aseptic cell culture system can reduce time and costs for storing, transporting, and using the aseptic cell culture system compared to at least some currently available cell culture devices.

As will be described in greater detail below, the aseptic cell culture system can include a cell culture device and at least one aseptic port integrated with the cell culture device. The cell culture device can include an interior chamber configured to contain, for example, cells and/or cell culture medium. In some embodiments, the cell culture system includes connecting ports configured to connect (e.g., permanently connect or releasably connect) to any one of the aseptic ports. For example, the aseptic port can include a genderless connecting interface that can couple with a genderless connecting interface of the connecting port. The connecting port and/or the aseptic port can include a membrane that prevents contaminants from entering a respective fluid pathway. The connecting port can include components such as a filter cartridge or fluid line connector for coupling a fluid line thereto.

In various embodiments of the present cell culture systems, an aseptic port includes a connecting component or an aseptic. The connecting component can be integrated or coupled to the port, such as a venting port, of a cell culture device to facilitate sterile venting, filtering and/or fluid transfer. The connecting component can include two coupling ends, a first coupling end for coupling to a port of the cell culture device and a second coupling end for coupling to tubing systems, filter assemblies venting assemblies, membranes, and other components for venting, sealing, filtering, transferring, or processing fluids in and out of the cell culture device. Each coupling ends of the connecting component can include a securing component or locking interface, element or mechanism that couples to tubing, filters, membranes, ports and other components for venting, filtering, transferring, or processing fluid. For example, connecting component at a first end can couple or attach directly or indirectly to cell culture device ports, and at a second end, couple or attach directly or indirectly to tubing assemblies, filter assemblies, venting assemblies and/or other fluid processing components by securing components, such as through opposing connector interface, snap-fit connections, barbed connections (barbed tubes/fittings), threaded engagement, adhesive, press-fit connections, ultrasonic welding or other securing components and techniques that facilitate easy, quick and/or aseptic connection. In exemplary embodiments, the connecting component is coupled to the cell culture device, ports, tubing, filters, membranes, and other components by permanent connections, connections that last through the life cycle of the cell culture device, or connections that last for a single use of the cell culture device (e.g., single culture run or one cell growth cycle within the device). As such, the aseptic cell culture system can be configured to enable breathing of the interior chamber and/or to enable introducing and/or removing of fluids while maintaining an aseptic environment within the aseptic cell culture systems herein disclosed.

Various embodiments of the aseptic cell culture system are described herein. For example, in some embodiments more than one aseptic port can be coupled to the cell culture device. Additionally, the cell culture device can include a multi-layer cell growth system and/or any number of a variety of cell or bio-material containers (e.g., trays, etc.) for storing and cultivating cells or bio-material. An example embodiment of the cell culture device is described in detail below.

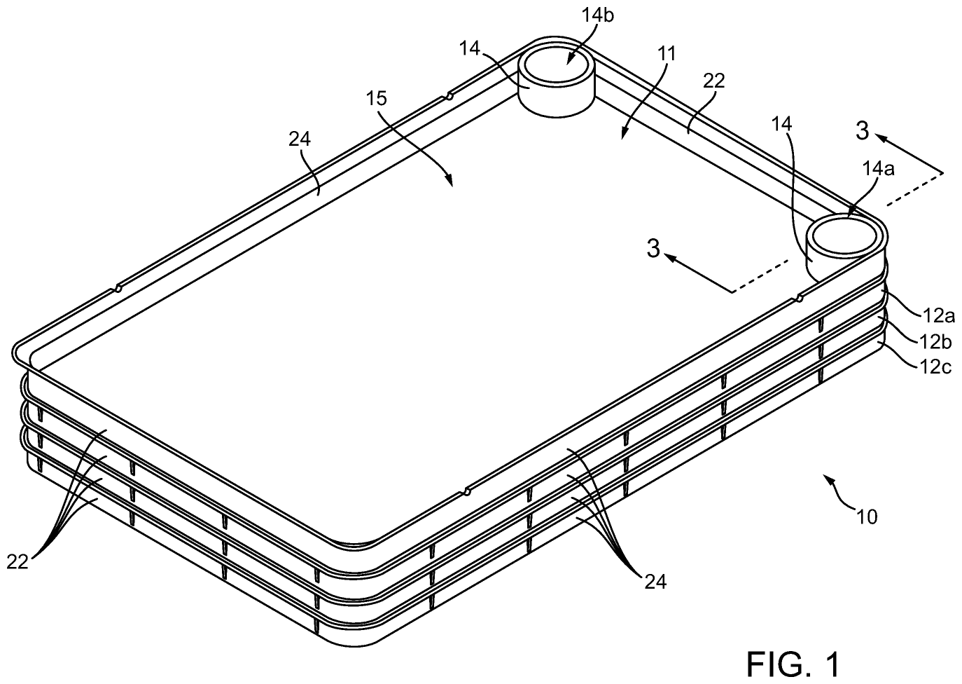

FIGS. 1-3 illustrate an embodiment of a cell culture device 10 comprised of a plurality of trays 12a, 12b, 12c that are each configured for the cultivation of cells. In some embodiments, the cell culture device 10 can include a lid 11 that is configured to cover the top tray, such as top tray 12a, as shown in FIGS. 1-3. The lid 11 can include one or more of the same or similar features as the trays and/or can include additional features that are not included in the trays. Each tray 12a, 12b, 12c can be stacked vertically with respect to another tray 12a, 12b, 12c, and the lid 11 can be stacked vertically on the top tray 12a. Although the number of trays 12a, 12b, 12c may vary, the illustrative embodiments include three trays and a lid.

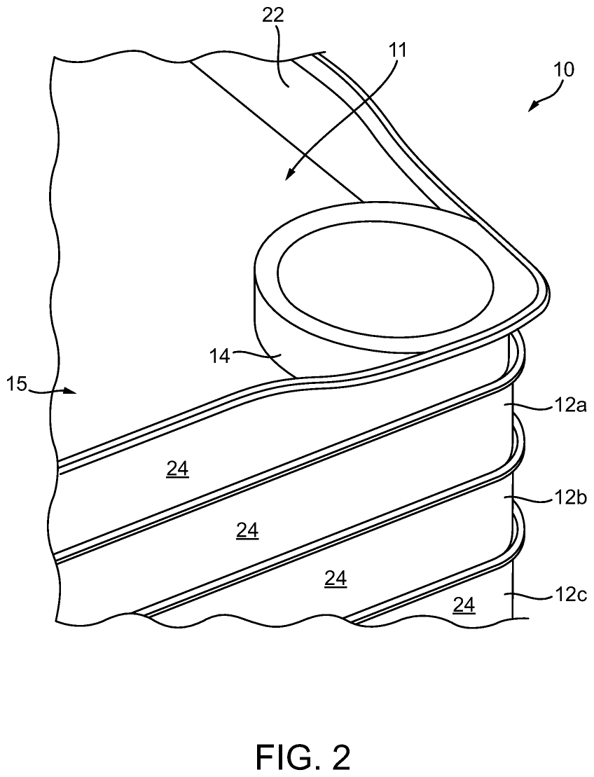

The trays 12a, 12b, 12c are configured to be in fluid communication with each other and form an interior chamber 15 having a total device volume that is the sum of each volume 13a, 13b, 13c of the respective tray 12a, 12b, 12c. Gas exchange between the total device volume of the interior chamber 15 and the environment outside the cell culture device 10 may occur via one or more openings or venting ports 14 (14a, 14b) along a wall defining a part of the interior chamber 15 of the cell culture device 10, as shown in FIG. 2.

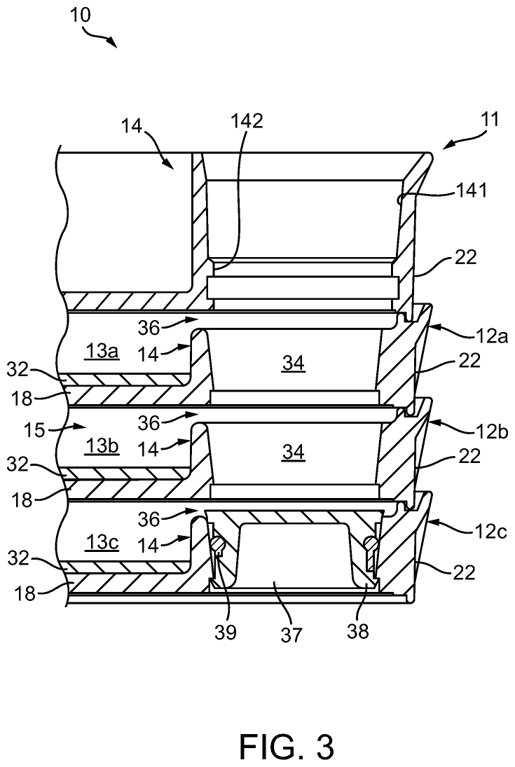

As shown in FIG. 3, each tray 12a, 12b, 12c includes a floor or bottom 18 defining a growth surface and at least one wall extending upwardly from the bottom 18 of the tray 12a, 12b, 12c. While the lid 11 and/or trays 12a, 12b, 12c may incorporate any suitable shape, including, for example, rectangular, square, round, circular, oblong, elliptical, polygonal, or trapezoidal, the illustrated cell culture device 10 is substantially rectangular and includes two substantially parallel end walls 22 and two substantially parallel side walls 24, which can extend upwardly from the bottom 18 of the tray 12 and/or along one or more sides of the lid 11. When stacked, the bottom 18 of one tray may provide a top or cover for an immediately adjacent tray. In some embodiments, a top tray may remain open to the environment outside the cell culture device 10 or the lid 11 may be used. Any number of trays can be stacked together to form the cell culture device 10, such as more than 10 trays, more than 50 trays, and more than 100 trays.

The lid 11 and/or trays 12a, 12b, 12c may be molded using a thermoplastic material, including, for example, polystyrene. Depending upon the material used, the thickness of the lid 11 and/or tray bottom 18 may vary but sufficient to prevent significant bowing of the lid 11 and/or tray bottom 18 when the cell culture device 10 is filled with an appropriate volume of culture medium 32, as shown in FIG. 3. For polystyrene, for example, a thickness of about 0.5 mm is recommended for trays having a culture surface area of about 632 cm 2 and configured to hold about 200 mL to about 300 mL of culture medium 32. Some embodiments may include trays having a surface area ranging from approximately 200 cm′ and 700 cm 2, but sizes outside this range are also contemplated.

In some embodiments, the cell culture device 10 may be constructed from a material that withstands sterilization, including, for example, sterilization by irradiation (beta or gamma radiation), steam autoclave, ethylene oxide, chemical disinfectants, or dry heat sterilization. In these or other embodiments, the cell culture device 10 may be made from a thermoplastic material and/or from a material that is formed, for instance, by injection molding. Examples of materials that are suitable for use in the present context include, for example, polyethylene, polypropylene, polystyrene, polycarbonate, polyurethane, polysulfone, polymethylpentene, polymethylmetacrylate, polyethyleneterepthtalate, polytetrafluoroethylene, or ABS (acrylonitrilbutadiene styrene). However, the examples given here are only exemplary in nature. A person skilled in the art would readily appreciate how to select other materials suitable for use in constructing the cell culture device 10. In some embodiments, the cell culture device 10 may be constructed by joining adjacent trays 12a, 12b, 12c and the lid 11 and using, for example, laser welding, ultrasonic welding, solvent bonding, or adhesive bonding (gluing) to secure the joined trays 12a, 12b, 12c and lid 11. Such adjoining of adjacent trays 12a, 12b, 12c and lid 11 can create sealed couplings between the trays and lid, thereby preventing unwanted fluid (e.g., liquid, air, gas, etc.) travel into and out of the cell culture device 10.

With reference now to FIG. 3, trays 12a, 12b, 12c are shown as including a volume of a liquid, such as a cell culture medium 32. The trays 12a, 12b, 12c are generally in fluid communication with one another via the venting ports 14 that, being a portion of a tray 12a, 12b, 12c, can define a fluid path or channel 34 between and among the trays 12a, 12b, 12c. An opening 36 is formed between the venting port 14 of each tray 12a, 12b, 12c and the above and immediately adjacent tray or lid 11 and provides fluid communication between the volumes 13a, 13b, 13c of the trays 12a, 12b, 12c. In this way, gases may be exchanged between the cell cultures within each tray and a more uniform growth environment can be achieved. The channel 34 can include a lower-most end 37 that opposes the venting port 14 of the top tray 12a and/or lid 11 and is within the lower-most tray 12c. In that regard, the lower-most tray 12c may include a stopper 38 within the respective venting port 14 to seal the lower-most end 37 of the channel 34 and to prevent inadvertent loss of the culture medium 32 and/or reduce the risk of microbial contamination. The stopper 38 may include one or more ribs, at least one thread, an O-ring 39, or other devices, with or without adhesives, for forming a seal with the venting port 14.

The cell culture device 10 can include a sealed interior chamber 15 such that only the venting ports 14 (e.g., along the lid 11) can provide a fluid pathway between the interior chamber 15 and the environment outside of the cell culture device 10. The device 10 can include any number of venting ports 14, such as along the lid 11, and one or more of the venting ports 14 can be sealed, such as with a stopper 38 to assist with containing the contents (e.g., cells, cell culture medium 32, etc.) of the cell culture device 10 in the interior chamber 15 while also assisting with keeping contaminants out of the interior chamber 15.

An important aspect of storing and treating the contents of the cell culture device 10 is to maintain an aseptic environment in the interior chamber 15, such as not to contaminate or disrupt the contents (e.g., contaminate or disrupt cell culturing). As such, the aseptic ports and aseptic cell culture systems described herein can optimize cell growth (e.g., by providing improved air vent filtration), facilitate easy connection of components to the cell culture device, prevent fluid leaks, and assist with efficiently and effectively maintaining a sterile and/or aseptic environment in the inner chamber 15 for efficiently and effectively storing, transporting, and using cell culture devices (e.g., maintain and cultivate various cells and biological mediums, etc.). Various embodiments of the aseptic ports and aseptic cell culture systems are described in detail below.

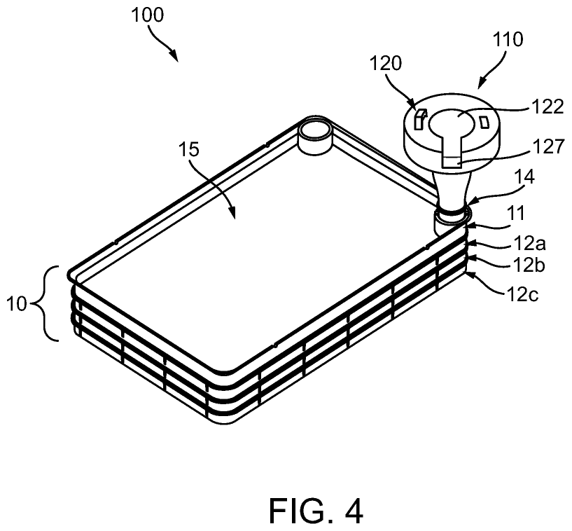

FIG. 4 illustrates an exemplary aseptic cell culture system 100 including an aseptic port 110 coupled to one of the venting ports 14 of an exemplary cell culture device 10. The aseptic port 110 can provide a fluid pathway for allowing the cell culture device 10 to effectively breathe and connect to various components. The aseptic port 110 can be integrated with the venting port 14 such that a user can receive the aseptic culture system 100 with the aseptic port 110 permanently attached to the cell culture device 10. For example, the aseptic port 110 can be integrated with the cell culture device 10 via one or more of mechanical couplings (e.g., locking mechanism), threaded engagement, an adhesive, snap-fit connection, ultrasonic welding, etc. For example, the aseptic port 110 can include at least a part of a locking mechanism that secures the aseptic port 110 to the cell culture device, such as within the venting port 14. The secure coupling between the aseptic port 110 and the cell culture device 10 can assist with ensuring a sterile and/or aseptic environment is maintained in the interior chamber 15, such as before and during use of the cell culture system 100. In some embodiments, the locking engagement between the aseptic port 110 and the venting port can be releasable.

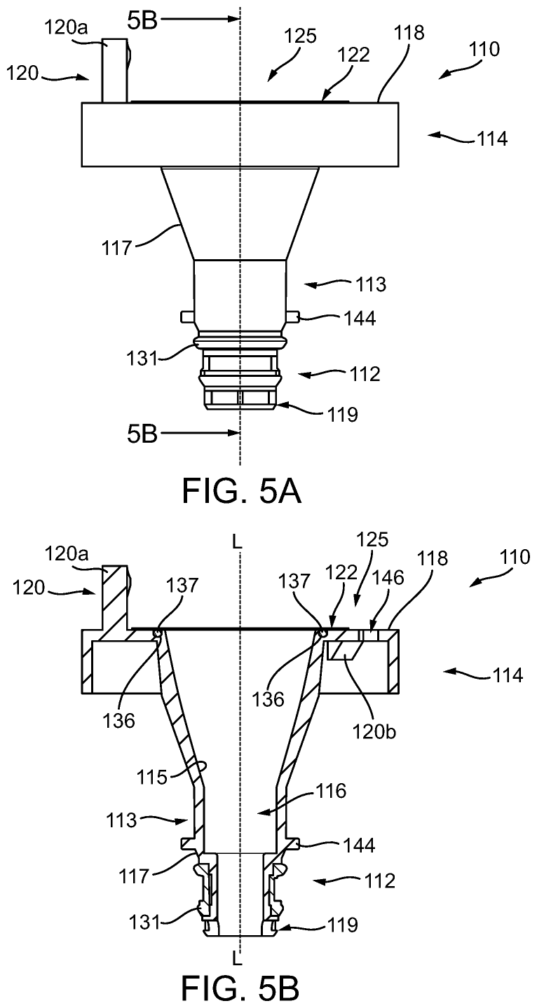

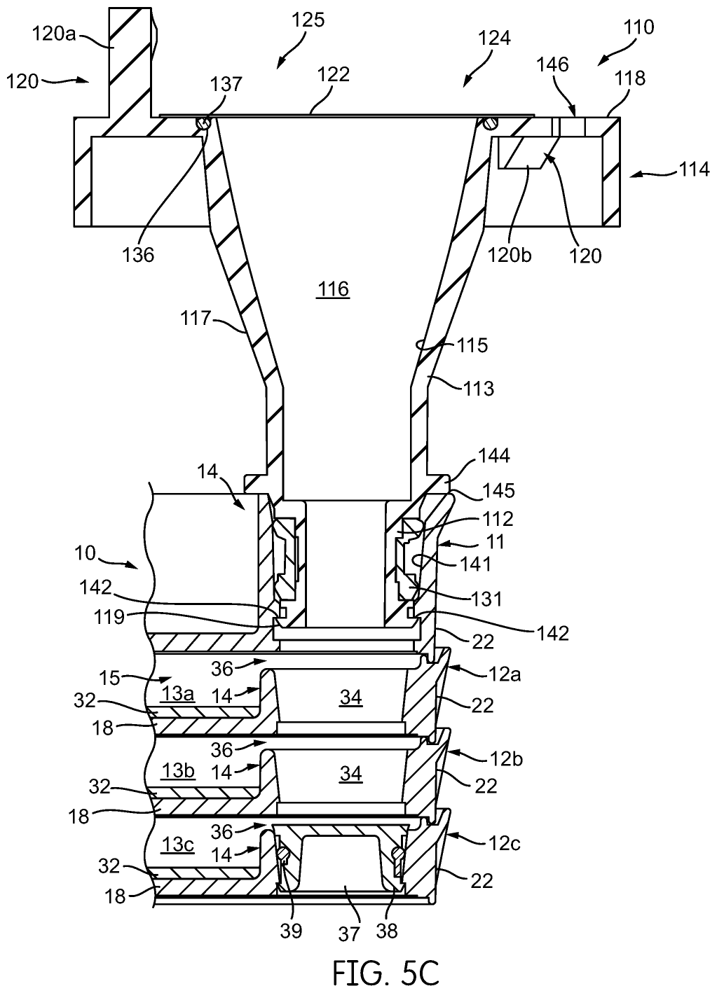

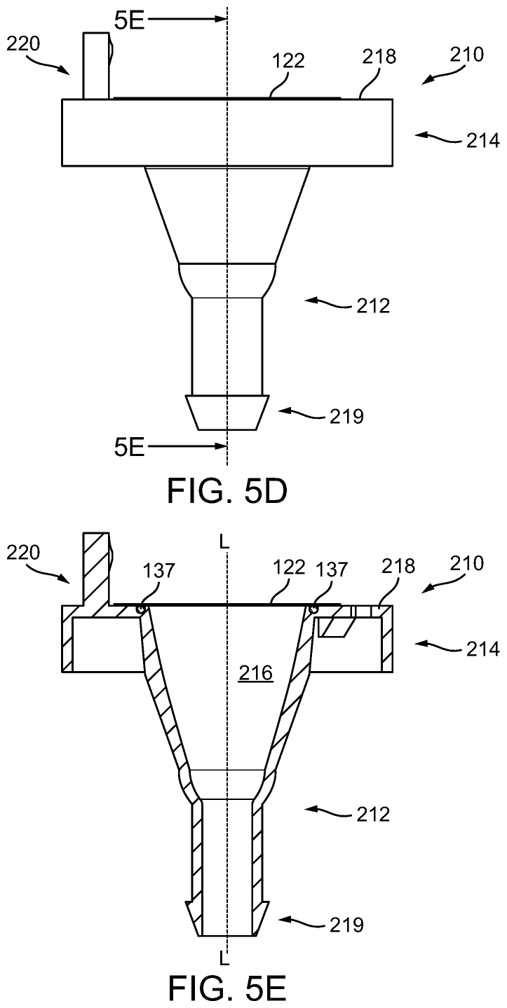

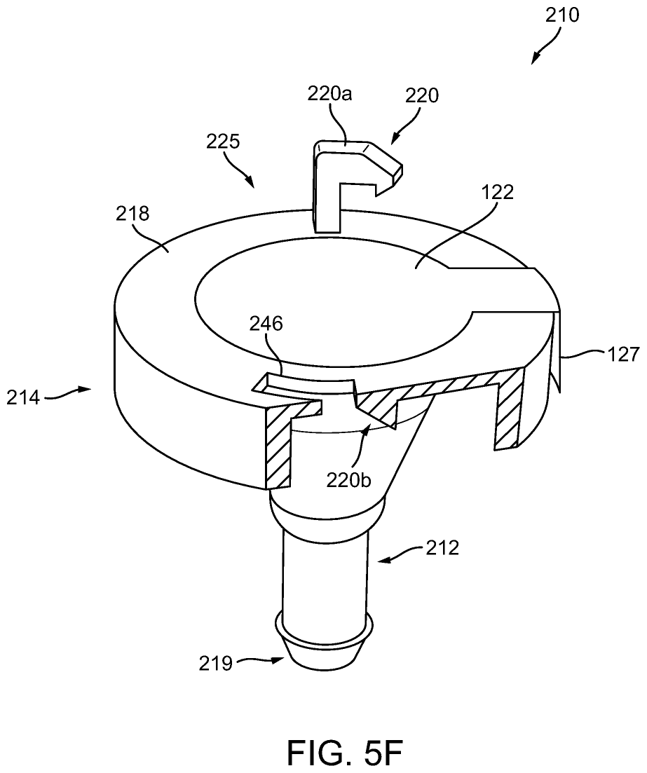

In various embodiments of the present cell culture systems, an aseptic port 110, including connecting component 120 (e.g., aseptic connector), can be integrated or coupled to a venting port 14 of a cell culture device 10 to facilitate sterile gas venting, and sterile fluid sealing, filtering, transfer and/or processing. The connecting component 120 can include two coupling ends, a first coupling end for coupling to the vent port 14 of the cell culture device 10 and a second coupling end for coupling to tubing systems, filter assemblies, membranes, other ports, other connectors, and/or other components for venting, filtering, transferring, or processing gas or fluid in and out of the cell culture device 10. Coupling ends of the connecting component 120 can include a securing component (shown in FIGS. 5A-C) or locking interface that couples to tubing, filters, membranes, ports, other connectors, and other components for venting, filtering, sealing, transferring, or processing fluid. With this configuration, connecting component 120 can couple or attach cell culture device ports to tubing assemblies, filter assemblies, venting assemblies and/or other fluid processing components by securing component, such as through opposing connector interface, snap-fit connections, barbed connections (barbed tubes/fittings), threaded engagement, adhesive, press-fit connections, ultrasonic welding or other securing components and techniques that facilitate easy, quick and/or aseptic connection with the cell culture device 10.

As will be described in greater detail below, the aseptic cell culture system 100 can prevent or at least reduce contamination of the interior chamber 15 of the cell culture device 10 (shown in FIGS. 1-3), which can result in reduced time and costs for users of the cell culture system 100. For example, reducing contamination of the interior chamber 15 of the cell culture device 10 can reduce time and costs associated with sterilizing and verifying sterile conditions of the cell culture system 100 prior to use. For example, in some embodiments the aseptic port can include and/or be coupled to a device having an integrated filter that is optimized and can assist with providing enhanced oxygen transfer within the cell culture device 10 to optimize cell growth.

As shown in FIG. 4, the aseptic port 110 can include a connecting component 120 attached at a first coupling end to vent port 14 and at a second coupling end to a membrane 122 that can prevent contaminants from passing through the aseptic port 110 and into the interior chamber 15. Additionally, the aseptic port 110 can allow for a variety of connections and interactions with the cell culture device 10 while maintaining an aseptic environment in the interior chamber 15. This can allow various uses of the aseptic cell culture system 100 for cultivating and storing a variety of cells and/or cell culture medium while requiring less sterilization by the user, as well as reducing or preventing contamination of the interior chamber 15 and its contents.

Various embodiments of the aseptic port 110 are described herein that can be integrated or securely coupled, such as during manufacturing, to one or more of a variety of cell culture devices 10. In exemplary embodiments, the aseptic port 110 can be made by integrating components of the aseptic port 110 via mold or extrusion as one unitary piece or by coupling more than one component or piece of the aseptic port 110 to form one part during manufacturing. The aseptic port 110 can also be integrated or coupled to an outlet or venting port 14 of the cell culture device 10 (e.g., the venting port 14 of the lid 11) during manufacturing. Additionally, more than one aseptic port 110 can be coupled to the device 10 and without departing from the scope of this disclosure. Each aseptic port 110 can provide a variety of functions to allow the cell culture device 10 to provide optimal oxygen mass transfer to cells or growth medium, achieve a pressure within a desired pressure range, and aseptically couple various components to the aseptic cell culture system 100.

FIGS. 5A and 5B illustrate an embodiment of the aseptic port 110 including a first coupling end 112 and a second coupling end 114. As shown in FIG. 5B, the aseptic port 110 can include a fluid pathway 116 that extends through the aseptic port 110 between the first coupling end 112 and the second coupling end 114. The first coupling end 112 can include an elongated shaft or tubular body 113. The tubular body 113 can include an inner wall 115 that defines a part of the fluid pathway 116. The tubular body 113 can include an outer wall 117 with one or more securing components 119 that assist with securing the aseptic port 110 to the venting port 14.

For example, as shown in FIG. 5B, the securing component 119 can include one or more extruded connecting components (e.g., barb, teeth, shoulder, ledge, etc.) configured to engage and secure with an inner wall engagement component 142 of the venting port 14. For example, the inner wall engagement component 142 can include one or more of a groove, a shelf, an indent that engages with the securing component 119 of the port 110, as shown in FIGS. 3 and 5C. The inner wall engagement component 142 and/or the securing component 119 can deform and/or allow the securing component 119 to travel past the inner wall engagement component 142 as the first coupling end 112 is being inserted into the venting port 14. The first coupling end 112 can be inserted into the venting port 14 until one or more flanges 144 extending from the outer wall 117 of the aseptic port 110 engage and/or contact a top surface 145 of the port 14 thereby preventing further travel. Once inserted, the inner wall engagement component 142 can prevent the first coupling end 112 from being removed from the venting port 14 (e.g., due to a locked engagement between the securing component 119 and the inner wall engagement component 142). Such locked coupling can integrate the aseptic port 110 with the cell culture device 10 and prevent uncoupling of the aseptic port 110 from the cell culture device 10, thereby forming an embodiment of the aseptic cell culture system 100. As shown in FIG. 5C, the integration and locked coupling of the aseptic port 110 with the cell culture device 10 to form the aseptic cell culture system 100 can include restricted or prevented longitudinal movement of the aseptic port 110 relative to the venting port 14, such as due to the flange 144 preventing further insertion of the first coupling end 112 into the venting port 14 and the locked engagement between the securing component 119 and the inner wall engagement component 142 preventing retraction or removal of the first coupling end 112 relative to the venting port 14. In some embodiments, the locked coupling between the aseptic port 110 and the cell culture device is releasable, such as for releasing the coupling between the aseptic port 110 and the cell culture device 10, as needed.

As shown in FIG. 5C, when the aseptic port 110 is integrated with the venting port 14, the fluid pathway 116 of the aseptic port 110 is in fluid communication with at least one opening 36 and the fluid path or channel 34 of the cell culture device 10. The channel 34 is part of and in fluid communication with the interior chamber 15 of the cell culture device 10. In some embodiments, the first coupling end 112 can include at least one O-ring 131 coupled to and extending around the outer wall 117, which can assist with forming a fluidic seal between the aseptic port 110 and the venting port 14 (e.g., the inner wall 141 of the venting port 14) of the cell culture device 10, thereby limiting fluid flow into and out of the interior chamber 15 via the fluid pathway 116 of the aseptic port 110. In some embodiments, an adhesive or other seal formation (e.g., ultrasonic welding, heat sealing, etc.) can assist with forming a fluidic seal between the aseptic port 110 and the cell culture device 10 thereby limiting fluid flow into and out of the interior chamber 15 via the fluid pathway 116 of the aseptic port 110. Similarly, if more than one aseptic port 110 is coupled to the cell culture device 10, fluidic seals between each aseptic port 110 and the cell culture device 10 can limit fluid passage into and out of the interior chamber 15 through the fluid pathways 116 of the more than one aseptic port 110.

In some embodiments, the fluid pathway 116 can have a diameter adjacent the distal end 124 that is approximately 5 millimeters (mm) to approximately 40 mm, such as approximately 25 mm. A proximal end of the fluid pathway 116 adjacent the distal end of the second coupling end 112 can have a diameter that is approximately 5 mm to approximately 10 mm, such as approximately 7 mm.

The permanent coupling between the aseptic port 110 and cell culture device 10, as well as limiting airflow into and out of the cell culture device 10 through the aseptic port 110 can contribute to maintaining the desired sterile and/or aseptic environment in the interior chamber 15. As will be described in greater detail below, the aseptic port 110 can also provide secure coupling with other connecting ports and devices, such as for providing various filtrations, introducing fluids into the interior chamber 15, and/or removing fluids out of the interior chamber 15, while maintaining an aseptic environment in the interior chamber 15.

As shown in FIGS. 5A and 5B, the second coupling end 114 of the aseptic port 110 can include at least one connecting component 120 along and/or adjacent to a planar surface 118. The planar surface 118 can have a flat circular shape that extends approximately perpendicular to a longitudinal axis L of the aseptic port 110. The planar surface 118 can have any of a variety of shapes, such as square, hexagonal, etc., without departing from the scope of this disclosure. As shown in FIGS. 5A and 5B, the connecting component 120 can include a first connecting component 120a (e.g., L-shaped hook, etc.) that extends out from the planar surface 118 and a second connecting component 120b that includes a depression or through hole 146 along the planar surface 118. The first connecting component 120a can be positioned at least partly along a same plane or axis as the second connecting component 120b, as well as positioned a same distance from the longitudinal axis of the aseptic port 110. This can allow another same or similar aseptic port 110 to securely engage with each other, as will be described further below. Any of a variety of features can be included in the connecting component 120 for coupling aseptic ports 110 without departing from the scope of this disclosure. The connecting component 120 shown in FIG. 5B, including the first connecting component 120a and the second connecting component 120b along and/or adjacent the planar surface 118, can form a genderless connecting interface 125 at the second coupling end 114 of the aseptic port 110 that can securely couple to a variety of connecting ports and/or devices having complementing connecting components. The connection between aseptic ports 110 and/or other connecting ports or devices can include a fluidic seal that prevents fluids (e.g., liquid, gas, air) from escaping along the connection. As shown in FIGS. 5B and 5C, the planar surface 118 can include a circular depression 136 that can receive a gasket ring 137. The circular depression 136 with gasket ring 137 can encircle the distal end 124 of the fluid pathway 116 thereby providing a fluidic seal around the distal end 124 of the fluid pathway 116 when the aseptic port 110 is coupled to another connecting port having a planar surface connecting interface. Other embodiments of connecting components 120 and fluidic seals are within the scope of this disclosure.

In some embodiments, the membrane 122 can be positioned across a cross-section of the fluid pathway 116, such as along one or more positions along the fluid pathway 116 for preventing contaminants from passing through the aseptic port 110. For example, the membrane 122 can be positioned along and/or releasably coupled to the planar surface 118 and cover a distal end 124 of the fluid pathway 116 and the gasket ring 137. For example, the membrane 122 can be at least partly sealed and/or connected to the gasket ring 137. The membrane 122 can be configured to allow air and gasses to pass through thereby allowing air and/or gasses to flow in and/or out of the interior chamber 15. This can allow the interior chamber 15 to maintain a pressure that is within a desired pressure range (e.g., does not damage the fluidic seals and/or structure of the cell culture device 10). Additionally, the membrane 122 can be formed of a material that assists with preventing particles and pathogens from passing through the membrane 122 thereby assisting with maintaining a sterile or aseptic environment in the inner chamber 15.

In some embodiments, the membrane 122 can be sealed or bonded to a part of the planar surface 118, such as using an adhesive to secure a part of the membrane 122 to the planar surface 118. Any number of a variety of securing components (e.g., adhesives, mechanical attachments, etc.) and/or methods (e.g., ultrasonic welding, etc.) can be implemented for securing the membrane 122 to the planar surface 118 and covering the distal end of the fluid pathway 116 for assisting with maintaining an aseptic environment in the interior chamber 15. As shown in FIG. 4, the membrane 122 can include an extension or tab 127 that extends away from the aseptic port 110 for allowing a user to grasp and separate the membrane 122 from the planar surface 118. For example, a user may want to remove the membrane 122 in a sterile environment to connect the cell culture device 10 to a fluid source for introducing a fluid into the interior chamber 15.

FIGS. 5D-5F illustrate an embodiment of a connecting port 210 that can couple to the aseptic port 110. The connecting port 210 can have at least some of the same or similar configurations and/or components as the aseptic ports 110 described herein. For example, the connecting port 210 can include an embodiment of the second coupling end 214 that is the same or similar to the second coupling end 114 of the aseptic port 110. For example, the second coupling end 214 can include a planar surface 218 along which a gasket ring 137 and membrane 122 are positioned along. As shown in FIG. 5E, the connecting port 210 can also include a connector fluid pathway 216 that extends along the length of the connecting port 210 between an embodiment of the first coupling end 212 and the second coupling end 214. The first coupling end 212 of the connecting port 210 can include an embodiment of the securing component, such as a tubing connector or barb 219, that can securely couple to a tubing or fluid line.

As shown in FIG. 5F, the connecting port 210 can include a same or similar genderless connecting interface 225 as the genderless connecting interface 125 of the aseptic port 125. The genderless connecting interface 225 of the connecting port 210 can securely couple to the genderless connecting interface 125 of the aseptic port 110. For example, the second coupling end 214 of the connecting port can include an embodiment of the connecting components 220 including first connecting component 220a (e.g., L-shaped hook, etc.) that extends out from the planar surface 218 and a second connecting component 220b that includes a depression or through hole 246 along the planar surface 218. The first connecting component 220a can be positioned at least partly along a same plane or axis as the second connecting component 220b, as well as positioned a same distance from the longitudinal axis of the aseptic port 110. For example, the genderless connecting surface 225 of the connecting port 210 can align associated first connecting component 220a with second connecting component 120b of aseptic port 110, as well align associated second connecting component 220b with first connecting component 120a of aseptic port 110. Once aligned, the planar surface 218 of the connecting port 210 can mate with the planar surface of the aseptic port 110 (e.g., as a result of mating of the connecting components 120, 220). In some embodiments, rotation of the connecting port 210 relative to the aseptic port 110 can allow for locking engagement of the first connecting components 220a, 120a, with the second connecting components 220b, 120b. For example, as shown in FIG. 5F, the first connecting component 220a can include an L-shaped hook that can engage and lock with an extruded feature of the second connecting component 220b (e.g., extending from a surface opposing the planar surface 218 and accessed via the through hole 246), including the L-shaped hook engaging and locking with the second connecting component 120b of the aseptic port 110 to form a locked coupling between the connecting port 210 and the aseptic port 110. Other connecting components and connections between the aseptic port 110 and connecting port 210 are within the scope of this disclosure.

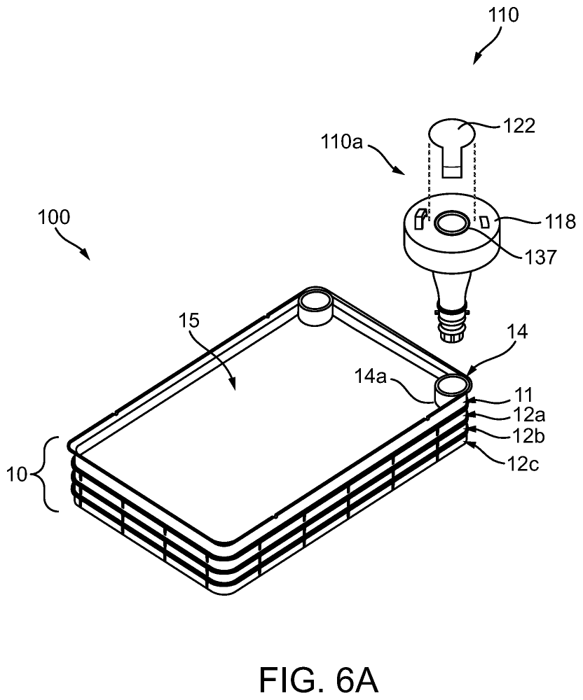

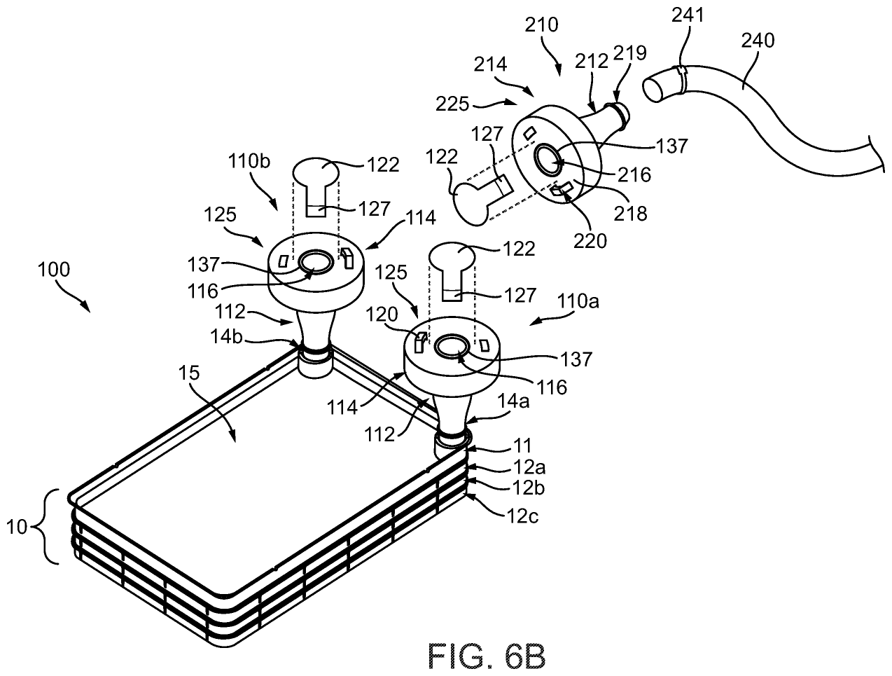

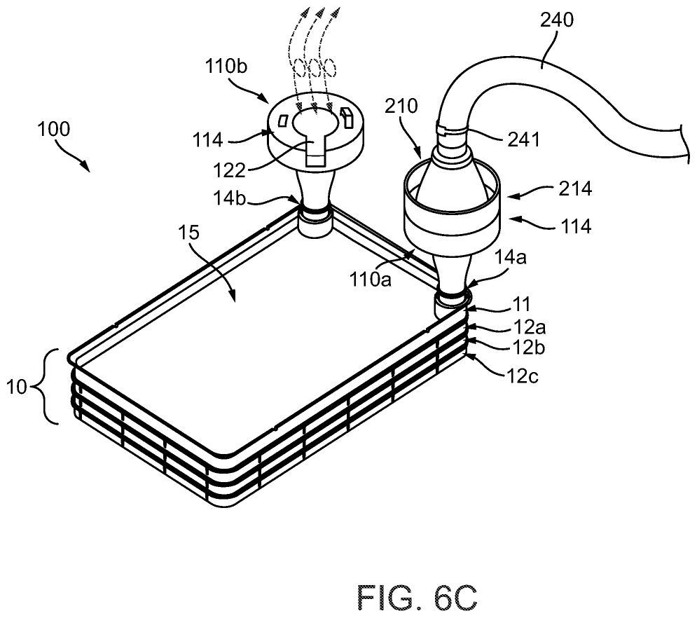

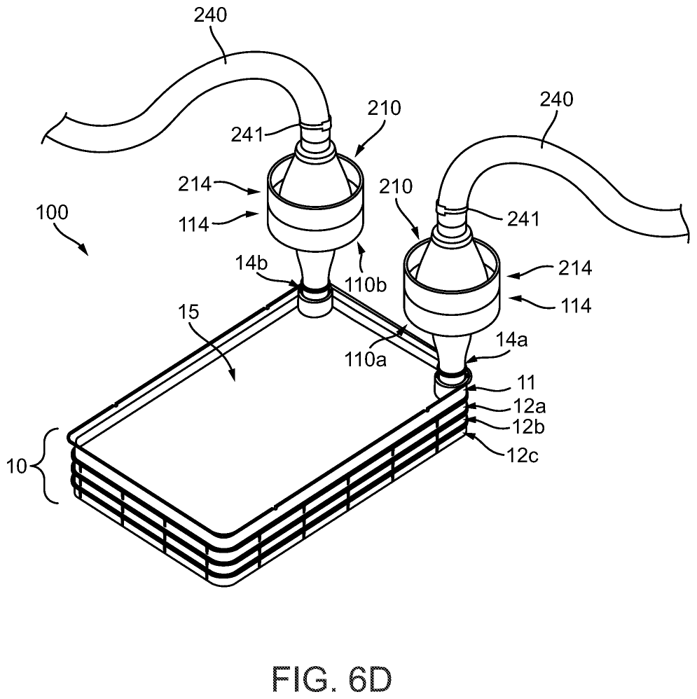

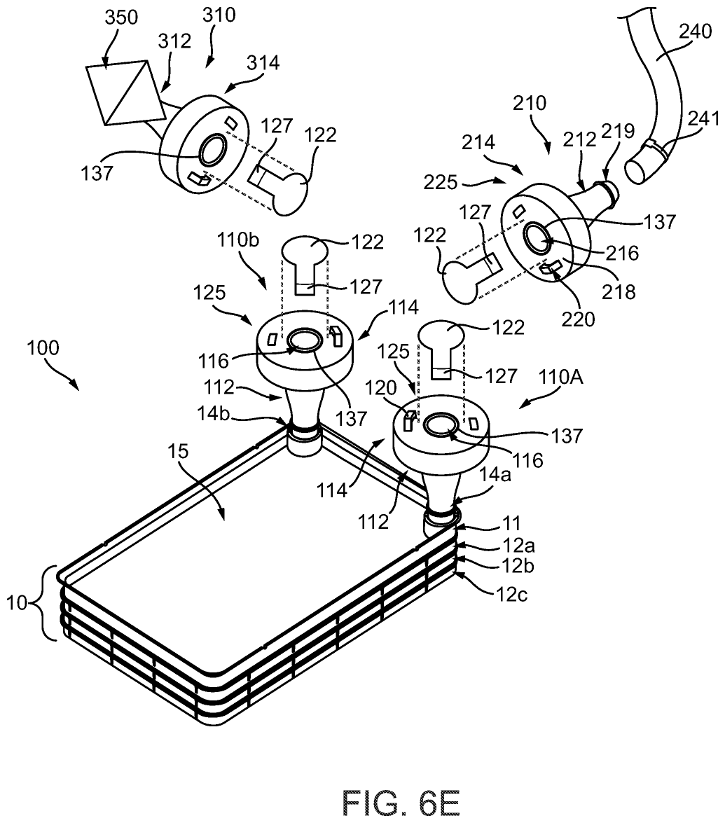

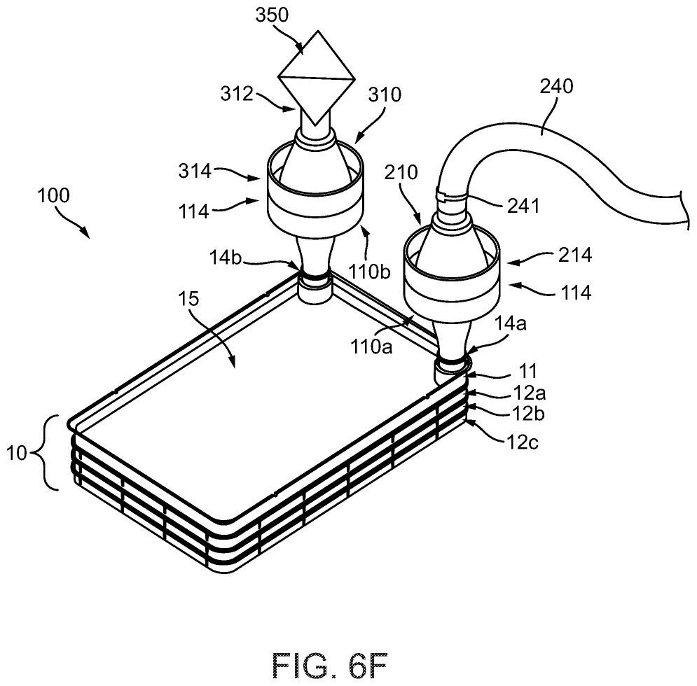

FIGS. 6A-6F illustrate various configurations and embodiments of the aseptic cell culture system 100, which can include at least one cell culture device 10 with at least one aseptic port 110 integrated with the cell culture device 10 for providing a sterile and/or aseptic environment in the interior chamber 15. As shown in FIGS. 6A, a first aseptic port 110a can be coupled to a first venting port 14a of the cell culture device 10 for integrating the first aseptic port 110a to the cell culture device 10 (as shown in FIG. 4). The first aseptic port 110a can include an embodiment of the membrane 122 coupled to the planar surface 118 of the first aseptic port 110a. For example, the cell culture device 10 and first aseptic port 110a including the membrane 122 and gasket 137 can be sterilized, such as during manufacturing, so that at least the interior chamber 15 is sterilized (e.g., prior to introduction of cells or cell culture medium 32 in the interior chamber 15).

Once sterilized, the aseptic cell culture system 100 can maintain a sterile environment in the interior chamber 15 due to the secure and fluidically sealed coupling of the first aseptic port 110a to the cell culture device 10, as well as due to the membrane 122 providing a protective covering over the fluid pathway 116 of the first aseptic port 110a. The membrane 122 can be formed out of one or more materials that allow fluid to pass through the membrane 122, such as air and/or various gases, while preventing contaminants from passing through the membrane 122 into the fluid pathway 116 and interior chamber 15. For example, the membrane 122 can be formed out of one or more of a polyethersulfone (PES) material, a Polyvinylidene Fluoride (PVDF) material, and a spun bonded sheet depth filter. As such, the aseptic port 110 can allow the interior chamber 15 to maintain a sterile environment prior to use (e.g., during shipping and/or storage), as well as allow the interior chamber 15 to breathe. Such ability to breathe and transfer gases, such as oxygen, to the cell media can optimize cell growth, allow the interior chamber 15 to achieve pressure equilibration and maintain pressure within a desired pressure range, which can prevent deformation and damage to the cell culture device 10 due to pressure differential inside and outside of the cell culture system 100.

For example, if no aseptic port 110 is coupled to the cell culture device 10 and the venting port 14 is sealed to maintain a sterile environment in the interior chamber 15, changes in environmental pressure (e.g., airline transportation, elevation changes, etc.) can deform and/or damage the cell culture device 10 (e.g., break seals, break welded joints, etc.), such as due to the interior chamber 15 being unable to vent or regulate the flow of gas in and out of the system 100 to equilibrate with the environment. Such deformation and damage can result in the cell culture device 10 being unusable and/or not sterile, thus requiring added cost and time for a user to sterilize and/or certify sterilization of the cell culture device 10 prior to use. However, if the venting port 14 is kept unsealed to allow for pressure equilibration of the internal chamber 15, the internal chamber 15 can be contaminated and unsterilized, thereby requiring added cost and time for a user to sterilize and/or certify sterilization of the device 10 prior to use.

As such, the aseptic cell culture system 100 described herein provides an efficient and effective way to aseptically seal the interior chamber 15 in order to maintain a sterile and/or aseptic environment in the interior chamber. Furthermore, such sterile and/or aseptic environment can be maintained from after the cell culture system 100 is sterilized through use of the cell culture system 100 (e.g., cell treatment, cell growth, cell cultivation, etc.). One or more of the aseptic port 110, the membrane 122, and the cell culture device 10 can be formed of a material that can withstand sterilization (e.g., x-ray, gamma irradiation, autoclave) and maintain sterile and/or aseptic conditions within the interior chamber 15 after sterilization.

As shown in FIG. 6B, a second aseptic port 110b can be coupled to a second venting port 14b of the cell culture device 10. As such, the first aseptic port 110a and the second aseptic port 110b can each provide a fluid pathway 116 into and out of the cell culture device 10 while maintaining a sterile and/or aseptic environment in the interior chamber 15. As shown in FIG. 6B, the first aseptic port 110a and the second aseptic port 110b can each include a membrane 122 coupled (e.g., sealed) to each planar surface 118.

For example, the aseptic cell culture system 100 can be manufactured and assembled to include two aseptic ports 110, such as shown in FIG. 6B, which can allow for more airflow in and out of the cell culture device 10. As such, more or less aseptic ports 110 can be coupled to the cell culture device 10 to achieve a desired flow rate in and/or out of the cell culture device 10. For example, fluid flow rate speeds in and/or out of an embodiment of the cell culture system 100 can be approximately 4 Liters/minute to at least approximately 6 Liters/minute. The aseptic cell culture system 100 can also be configured to couple to a variety of components, such as for allowing introduction and/or removal of a liquid from the cell culture device 10. For example, one or more connecting ports 210, such as shown in FIGS. 5D-5F and described above, can connect to one or more aseptic ports 110, tubing (e.g., fluid line), and/or other component (e.g. filters, etc.) while maintaining an aseptic environment in the interior chamber 15.

As shown in FIG. 6B, an embodiment of the connecting port 210 can be configured to couple to the first port 110a or second port 110b. As discussed above with respect to FIGS. 5D-5F, the connecting port 210 can have the same or similar configurations and/or components as the aseptic ports 110 described herein. For example, the first coupling end 212 of the connecting port 210 can include an embodiment of the securing component, such a tubing connector or barb 219, that can securely couple to a tubing or fluid line 240. As such, the barb 219 of the first coupling end 212 of the connecting port 210 can be inserted into an end of the tubing 240 thereby securely coupling the tubing 240 to the connecting port 210. A clamp or tie 241 can be secured around the end of the tubing positioned over the barb 219 to assist with securing the coupling between the tubing 240 and connecting port 210, as well as ensure a fluidic seal between the tubing 240 and the connecting port 210. For example, the tubing 240 can have an inner diameter that is approximately 0.3125 inch to approximately 0.375 inch.

The connecting port 210 can be permanently coupled or releasably coupled to the aseptic port 110, such as the first aseptic port 110a shown in FIG. 6C. As described above with respect to FIGS. 5D-5F, the second coupling end 214 of the connecting port 210 can include an embodiment of the planar surface 218 and the connecting components 220 such that the second coupling end 214 of the connecting port includes a genderless connecting interface 225 that can securely couple to the genderless connecting interface 125 of the aseptic port 110. Furthermore, the aseptic port 110 can include an embodiment of the gasket ring 137 along its associated planar surface 118 and/or the connecting port 210 can include an embodiment of the gasket ring 137 along its associated planar surface 218. As such, when the connecting port 210 is connected to the aseptic port 110 (e.g., via engagement of the connecting components 120 and 220), the one or more gasket rings 137 can assist with forming a fluidic seal between the planar surfaces 118 and 218 of the aseptic port 110 and the connecting port 210, respectively.

In some embodiments, prior to securely coupling the connecting port 210 to the aseptic port 110, the membranes 122 along the connecting port 210 and the aseptic port 110 can be removed. For example, the genderless connecting interface 225 of the connecting port 210 can be placed in contact and/or adjacent to the genderless connecting interface 125 of the aseptic port 110 such that the membranes 122 of the connecting port 210 and aseptic port 110 are in contact and positioned between the planar surface 218 of the connecting port 210 and the planar surface 118 of the aseptic port 110. The membranes 122 may then be removed between the planar surfaces 118, 218, such as by pulling on the tabs 127 of the membranes 122 in a direction perpendicular to the longitudinal axis L of the aseptic port 110. This can cause the membranes 122 to slide out between the planar surfaces 118, 218 and allow the connecting port 210 to securely couple to the aseptic port 110 to maintain a sterile environment in the aseptic cell culture system 100.

As shown in FIG. 6C, the first aseptic port 110a coupled to the connecting port 210 including tubing 240 can provide a fluid pathway for introducing one or more fluids and/or gases into the interior chamber 15. The second aseptic port 110b can allow for sufficient breathing (e.g., aeration) of the aseptic cell culture system 100. For example, when introducing fluids into the interior chamber 15 via the first aseptic port 110a, the second aseptic port 110b can release air to achieve desired pressure ranges within the interior chamber 15 (e.g., prevent damage to the cell culture device 10).

As shown in FIG. 6D, another connecting port 210 including connected tubing 240 can be coupled to the second aseptic port 110b. The tubing 240 can provide liquid or air/gas transport. As such, the first aseptic port 110a and associated connecting port 210 can provide a fluid pathway for liquid to be introduced into the interior chamber 15 or removed from the interior chamber 15, and the second aseptic port 110b and associated connecting port 210 can provide a fluid pathway for air/gas to be introduced into the interior chamber 15 or removed from the interior chamber 15.

FIGS. 6E-6F show an embodiment of the aseptic cell culture system 100 including the first aseptic port 110a integrated with the first venting port 14a, the second aseptic port 110b integrated with the second venting port 14b, and a first connecting port 210 including a connected tubing 240 for connecting to the first aseptic port 110a. Additionally, the aseptic cell culture system 100 can include an embodiment of a connecting port 310 with a connected filter 350. As shown in FIGS. 6E and 6F, the filter 350 can be positioned at or adjacent to an embodiment of the first coupling end 312 of the connecting port 310. The filter 350 is shown schematically in FIGS. 6E and 6F such that any number of a variety of filter embodiments can be integrated with an embodiment of the connecting port 310. Furthermore, the connecting port 310 can be connected and coupled to a variety of aseptic ports 110, such as any of the aseptic ports 110 described herein (such as shown in FIGS. 5A and 5B).

The connecting port 310 can include an embodiment of the second coupling end 314 that includes any one or more features described herein with regards to embodiments of the second coupling end, such as an embodiment of the gasket ring 137 (for assisting with forming fluidic seal when connected to aseptic port 110) and membrane 122 (e.g., for providing sterile and/or breathable barrier). The membrane 122 can be releasably coupled to the connecting port 310 and can be removed during coupling of the connecting port 310 to the aseptic port 110. The filter 350 of the connecting port 310 can provide filtered aeration, such as while the first aseptic port 110a and connecting port 210 coupled thereto provide a fluid pathway in communication with the interior chamber 15 (e.g., for introducing or removing fluids).

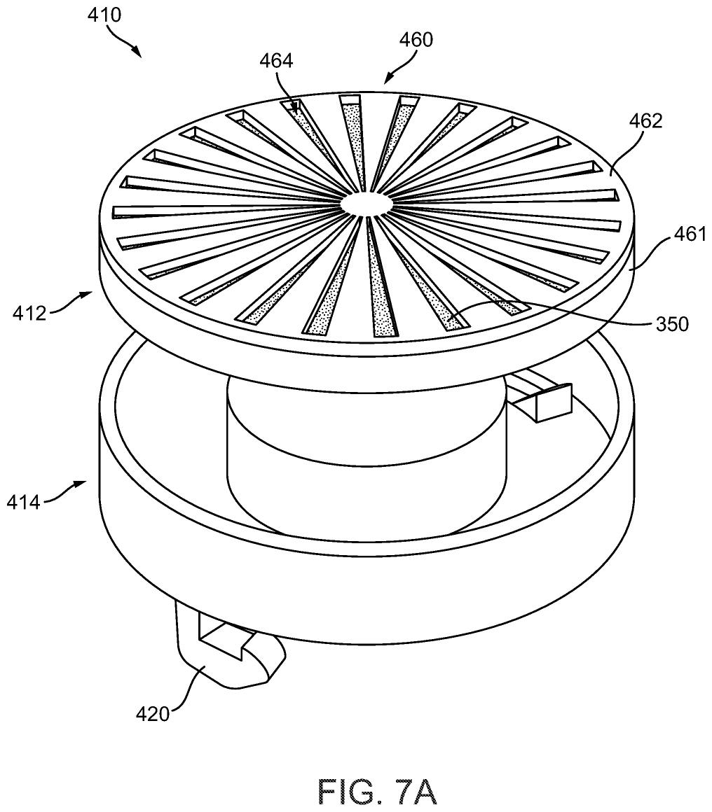

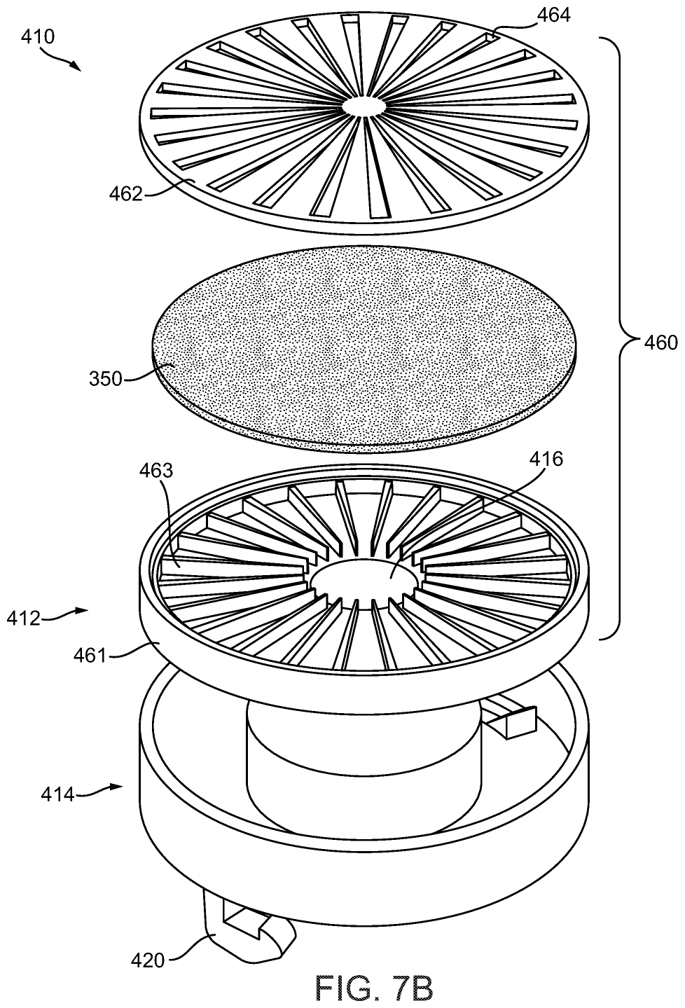

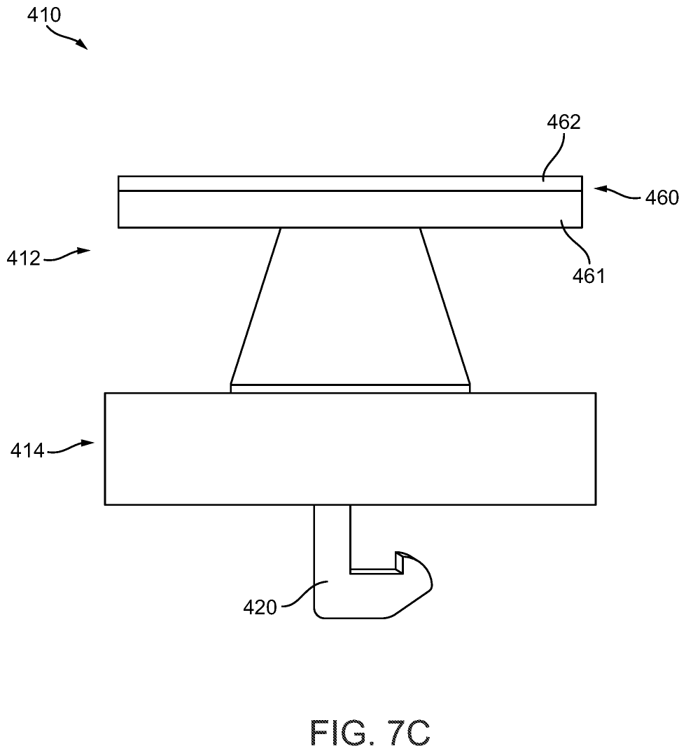

FIGS. 7A-7C show an embodiment of a connecting port 410 including a filter housing 460 containing an embodiment of the filter 350 (also shown as schematic embodiment in FIGS. 6E and 6F). The connecting port 410 including the filter housing 460 and filter 350 shown in FIGS. 7A-7F can be connected and coupled to a variety of aseptic ports 110, such as any of the aseptic ports 110 described herein (such as shown in FIGS. 5A and 5B).

As shown in FIGS. 7A and 7C, the filter housing 460 can be positioned at or adjacent to an embodiment of the first coupling end 412 of the connecting port 410. Additionally, the connecting port 410 can include an embodiment of the second coupling end 414 that includes any one or more features described herein with regards to embodiments of the second coupling end 414, such as an embodiment of the gasket ring 137 (FIG. 6E), membrane 122 (FIG. 6E), and/or connecting components 420 (e.g., L-shaped hook, etc.), as shown in FIG. 7C.

In some embodiments, the filter housing 460 can include a single part or be formed of more than one part, such as a housing base 461 and a housing lid 462, as shown in FIG. 7B. The housing base 461 can be coupled to the second coupling end 414 and include a part of an embodiment of the fluid pathway 416 extending therethrough. The housing lid 462 can include at least one through-hole 464, such as a plurality of through-holes 464 that are elongated and positioned radially along the housing lid 462. In some embodiments, the housing base 461 can include a plurality of vanes 463 that can assist with directing and/or controlling fluid flow (e.g., airflow) through the filter housing 460. As shown in FIG. 7B, the filter 350 can be positioned between the housing base 461 and housing lid 462. The housing lid 462 can be permanently (e.g., welded, bonded, etc.) or releasably secured (e.g., threaded engagement, snap-fit, friction fit, etc.) to the housing base 462. As such, the filter 350 can be permanently contained in the filter housing 460 or can be removable and/or replaceable.

During use, for example, airflow can travel along the fluid pathway 416, such as in the direction of the filter housing 460 and/or away from the filter housing 460. For example, the airflow can travel into the filter housing 460 through one or more of the plurality of through-holes 464 along the housing lid 462. The airflow can then flow through the filter 350 and then guided along and/or between vanes 463 along the housing base 461 towards and into the fluid pathway 416. Once in the fluid pathway 416, the airflow can travel through the second coupling end, such as into the interior chamber 15 (FIG. 6F) for equalizing pressure within the interior chamber 15. Such airflow into the interior chamber 15 can be free from one or more contaminants removed from the airflow by the filter 350.

In some embodiments, the filter 350 can be removed and/or replaced in the filter housing 460. For example, the housing lid 461 can be removably coupled to the housing base 462 such that the housing lid 461 can be uncoupled from the housing base 462 to allow the filter 350 to be removed and/or replaced. After the filter 350 is removed and/or replaced from the filter housing 460, the housing lid 461 can reattached to the housing base 462.

Other components and combinations of connecting ports for connecting to one or more aseptic ports integrated with the cell culture device are within the scope of this disclosure.

In some embodiments, a method of the aseptic cell culture system 100 includes introducing a cell culture medium (e.g., culture medium 32) to the interior chamber 15 of a cell culture device 10 of the cell culture system 100. The cell culture medium can be introduced through an embodiment of the aseptic port 110 integrated with the cell culture device 10. The method can also include removing, after processing the cell culture medium in the interior chamber 15, a volume of cell material from the aseptic cell culture system 100. The method can further include disposing, after removal of the volume of cell material, the aseptic cell culture system 100. As such, the aseptic cell culture system 100 can be a single-use, disposable system.

Exemplary methods of manufacturing and/or assembling the aseptic cell culture system 100 can include coupling and integrating two aseptic ports 110 with two venting ports 14 of the cell culture device 10. During manufacturing, membranes 122 can be secured (e.g., heat sealed, welded, adhered) to each aseptic port 110 thereby forming a protective containment of the interior chamber 15. The aseptic cell culture system 100 can then be sterilized thereby sterilizing the interior chamber 15. Sterilization of the aseptic cell culture system 100 can include one or more of irradiation, autoclave, ethylene oxide, chemical disinfectants, and dry heat sterilization. For example, gamma-irradiation of at least 50 kilogray (kGy) can be used to sterilize the aseptic cell culture system 100. The sterilized aseptic cell culture system 100 can then be provided to a user for use.

In some embodiments, the aseptic cell culture system 100 can be provided as a cell culture kit to a user, which can include the aseptic cell culture system 100 including two aseptic ports 110 integrated with two venting ports 14 of the cell culture device 10. The cell culture kit can also include a plurality of connecting ports (such as connecting ports 210 and 310) that can be coupled to and/or include various components, such as any of the components described herein (e.g., filter 350, tubing 240). Furthermore, any one of the plurality of connecting ports can be configured to be coupled to either of the aseptic ports 110 thereby allowing a user to efficiently assemble an aseptic cell culture system 100 that is most suitable for an intended use.

Although the manufacturing, assembly and kit examples are described as including two aseptic ports 110 integrated with the cell culture device 10, any number of aseptic ports 110 can be included without departing from the scope of this disclosure.

While the invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. Therefore, the invention in its broadest aspects is not limited to the specific details shown and described. The various components disclosed herein may be used in any combination necessary or desired for a particular application. Consequently, departures may be made from the details described herein without departing from the spirit and scope of the claims which follow.

In the descriptions above and in the claims, phrases such as “at least one of” or “one or more of” may occur followed by a conjunctive list of elements or components. The term “and/or” may also occur in a list of two or more elements or components. Unless otherwise implicitly or explicitly contradicted by the context in which it is used, such a phrase is intended to mean any of the listed elements or components individually or any of the recited elements or components in combination with any of the other recited elements or components. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited component or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further components and/or variations can be provided in addition to those set forth herein. For example, the implementations described above can be directed to various combinations and sub-combinations of the disclosed components and/or combinations and sub-combinations of one or more components further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. An aseptic cell culture system, comprising:
a cell culture device including an interior chamber and a first opening extending through a wall defining a part of the interior chamber; and
a first aseptic port including a first coupling end and a second coupling end, the first coupling end extending through the first opening and integrated with the wall, the first aseptic port including a fluid pathway in fluid communication with the interior chamber and extending between the first coupling end and the second coupling end, the second coupling end including a connecting component configured to allow one or more of a fluid line and a filter to be connected to the first aseptic port.

2. The aseptic cell culture system of claim 1, wherein the first aseptic port comprises a membrane positioned across a cross-section of the fluid pathway, the membrane configured to prevent contaminants from passing through the membrane.

3. The aseptic cell culture system of claim 2, wherein the membrane is positioned along a planar surface of the second coupling end and covers a distal end of the fluid pathway.

4. The aseptic cell culture system of claim 3, wherein the connecting component is positioned along the planar surface, the connecting component and the planar surface forming at least a part of a first genderless connecting interface of the first aseptic port.

5. The aseptic cell culture system of claim 4, wherein the aseptic cell culture system further includes a connecting port having a second genderless connecting interface that couples with the first genderless connecting interface of the first aseptic port.

6. The aseptic cell culture system of claim 5, wherein the connecting port includes a connector fluid pathway that is in fluid communication with the fluid pathway of the first aseptic port when the connecting port is coupled to the first aseptic port.

7. The aseptic cell culture system of claim 6, wherein the connecting port includes a tubing connector configured to couple to a tubing for allowing fluid to flow into the interior chamber or out of the interior chamber when the connecting port is coupled to the first aseptic port.

8. The aseptic cell culture system of claim 7, wherein the fluid is one or more of a gas, air, liquid, and cell culture medium.

9. The aseptic cell culture system of claim 6, wherein the connecting port includes a filter positioned along the connector fluid pathway for filtering fluid flowing into and/or out of the first aseptic port connected to the connecting port.

10. The aseptic cell culture system of claim 1, wherein the second coupling end forms a locked engagement with the wall to prevent uncoupling of the first aseptic port from the cell culture device.

11. The aseptic cell culture system of claim 10, wherein the locked engagement is formed by one or more of an adhesive and a mechanical coupling between the first aseptic port and the cell culture device.

12. The aseptic cell culture system of claim 2, wherein the first aseptic port, the membrane and the cell culture device are formed of a material that withstands gamma-irradiation of at least 50 kilogray (kGy).

13. The aseptic cell culture system of claim 1, wherein the cell culture device includes a second aseptic port extending through a second opening of the cell culture device thereby providing a second fluid pathway for liquid and/or gas to flow either in or out of the interior chamber.

14. The aseptic cell culture system of claim 1, wherein the cell culture device is a multi-layer cell growth system comprising a plurality of cell culture trays.

15. A method of manufacturing an aseptic cell culture system, comprising:
coupling a first aseptic port to a first opening of a cell culture device to form the aseptic cell culture system, wherein the coupling comprises forming a locked engagement and a fluidic seal between the first aseptic port and the cell culture device; and
sterilizing the aseptic cell culture system.

16. The method of claim 15, wherein the sterilizing includes one or more of irradiation, autoclave, ethylene oxide, chemical disinfectants, or dry heat sterilization.

17. The method of claim 15, further comprising coupling a second aseptic port to a second opening of the cell culture device.

18. An aseptic cell culture system kit, comprising an aseptic cell culture system, comprising:
a cell culture device including an interior chamber and a first opening extending through a wall defining a part of the interior chamber; and
a first aseptic port including a first coupling end and a second coupling end, the first coupling end extending through the first opening and integrated with the wall, the first aseptic port including a fluid pathway in fluid communication with the interior chamber and extending between the first coupling end and the second coupling end, the second coupling end including a connecting component configured to allow one or more of a fluid line and a filter to be connected to the first aseptic port.

19. The aseptic cell culture system kit of claim 18, wherein the second coupling end of the first aseptic port forms a locked engagement with the wall to prevent uncoupling of the first aseptic port from the cell culture device.

20. The aseptic cell culture system kit of claim 19, wherein the locked engagement is releasable.
